(19) United States
(12) Patent Application Publication
(10) Pub. No.: US 2006/0154328 A1
(43) Pub. Date: Jul. 13, 2006

(54) Ionic liquids

(76) Inventors: Neil C Bruce, York (GB); Adam J Walker, Lincs (GB)

Correspondence Address: CONNOLLY BOVE LODGE & HUTZ, LLP, P O BOX 2207, WILMINGTON, DE, 19899, US

(21) Appl. No.: 10/541,670

(22) PCT Filed: Jan. 7, 2004
(86) PCT No.: PCT/GB04/00014

§ 371 Date: Dec. 30, 2005

(30) Foreign Application Priority Data
Jan. 10, 2003 (GB) 0300595.6

(51) Int. Cl. C12P 1/00

(52) U.S. Cl. 435/41

(57) ABSTRACT

This invention relates to ionic liquids and their use as solvents in biocatalysis. According to a first aspect of the invention there is provided a method of carrying out an enzyme-catalysed reaction comprising providing a liquid reaction medium which comprises an ionic liquid including an ion which comprises a functional group selected from the group consisting of alkenyl, hydroxyl, amino, thio, carbonyl and carboxyl groups, providing in the liquid reaction medium an enzyme and a substrate for the enzyme, and allowing reaction of the substrate to occur.

This invention relates to ionic liquids and their use as solvents in biocatalysis.

Traditionally, the fine chemical and pharmaceutical industries have relied on chemical transformations in organic solvents for the synthesis of desired products. Although enzymes have many properties making them ideal catalysts, the use of biocatalysis in industry has been limited by the requirement for conventional enzyme-based systems to operate in aqueous solution, whilst many of the key substrates and products desired by the fine chemical and pharmaceutical industries are very poorly soluble in water. Furthermore, water may elicit the hydrolysis of sensitive chemical species. Enzymes and enzyme cofactors are generally inactive in the organic solvents traditionally used for synthesis, being either insoluble in or denatured by these media.

Therefore, it would be desirable to be able to provide a solvent in which both enzymes and cofactors are active and in which a wide range of substrates and reaction products are soluble.

Ionic liquids are compounds which are composed entirely of ions but which have a melting point below ambient temperature. Ionic liquids have been known since the beginning of the 20^{th} century. They can be formed when relatively large molecules are used as the base and/or the acid to form an ionic salt. By using a large base or acid, the degree of order of the resulting salt can be reduced and the melting point lowered to a point where the resultant salt is liquid at ambient temperature. The delocalisation of the charge on the ion is also an important factor in determining the melting point of the resulting salt.

The use of ionic liquids as solvents for enzyme-catalysed reactions has been reported in cases where the enzyme is a particularly robust species. In many of these cases, the enzyme has also been shown to be active in molecular organic solvents. See (a) J. A. Laszlo and D. L. Compton, “Chymotrypsin-catalysed Transesterifications of Phenylalanine Esters in Ionic Liquids and Supercritical Carbon Dioxide”, Biotechnol. Bioeng., 2001, 75, 181-186, which requires either added water or supercritical carbon dioxide for activity and (b) R. Madeira Lau, S. Van Rantwijk, K. R. Seddon and R. A. Sheldon, “Lipase-Catalysed Reactions in Ionic Liquids”, Org. Lett., 2000, 2(26), 4189-4191. These enzymes shown to have activity in ionic liquids were non cofactor-dependent enzymes.

Enzyme-catalysed reactions in the presence of ionic liquids have also been successfully demonstrated in multiphase solvent systems. For example, the ionic liquid 1-butyl-3-methylimidazolium hexafluorophosphate has been used to replace organic solvents in multiphase bioprocessing operations. See S. G. Cull, J. D. Holbrey, V. Vargas-Mora, K. R.-Seddon and G. J. Lye, “Room-Temperature Ionic Liquids as Replacements for Organic Solvents in Multiphase Bioprocess Operations”, Biotech. Bioeng., 2000, 69, 227-233. Erbeldinger et al, in “Enzymatic Catalysis of Formation of Z-aspartame in Ionic Liquid”, Biotechnol. Prog., 2-000, 16 (6), 1129-1131, also disclose use of 1-butyl-3-methylimidazolium hexafluorophosphate as a solvent but say that water is essential for activity.

Cofactor-dependent enzyme systems are complicated by the fact that the critical redox processes which occur between enzyme and cofactor often require a polar, protic environment in which hydrogen-bonding is possible. This is normally provided by water. In industry, cofactor-dependent enzymes can only be used economically with the aid of some form of cofactor recycling system, due to the prohibitive cost of cofactors such as the nicotinamide cofactors. This generally requires the use of a second enzyme.

Cofactor-dependent enzymes have in the past only shown activity in ionic liquids within a biphasic system of an apolar-hydrophobic ionic liquid (containing the substrate) and water (containing most of the enzyme and cofactor and acting as a solvent for the redox reaction). In this case the actual enzyme-catalysed biotransformation occurs either in the water layer through-phase transfer or at the ionic liquid/water interface (see N. Kraftzik, P. Wasserscheid and U. Kragl, “Use of Ionic Liquids to Increase the Yield and Enzyme Stability in the Galactosidase Catalysed Synthesis of N-Acetyllactosamine”, Org. Proc. Res & Dev., 2002 (in print)).

As a further example, EP 1205555-A discloses the use of a reaction medium comprising an ionic liquid for a series of enzyme-catalysed reactions. The ionic liquids disclosed in the examples are standard ionic liquids of the types used in the references mentioned above, namely comprising cations based on nitrogen-containing compounds modified with alkyl substituents and anions such as hexafluorophosphate, tetrafluoroborate, methane sulfonate, nitrate, benzoate, trifluoromethanesulfonate and bis-(trifluoromethylsulfonyl)-imidate. Reactions utilising various enzymes are disclosed. In one example, a cofactor-dependent enzyme is used to carry out a reaction in a solvent comprising 25% to 75% ionic liquid and 75% to 25% buffer solution.

We have now found that by modifying the component ions of ionic liquids it is possible to produce ionic liquids compatible with single-phase biocatalysis, which are particularly suitable for use with cofactor-dependent enzymes. By selectively modifying the ionic liquids it is possible to make these solvents more biocompatible but without greatly increasing the order or the ionic weight of the ionic liquid and therefore without elevating the melting point above the temperature required for the reaction to occur.

According to a first aspect of the invention, there is provided a method of carrying out an enzyme-catalysed reaction comprising

providing a liquid reaction medium which comprises an ionic liquid including an ion which comprises a functional group selected from the group consisting of alkenyl, hydroxyl, amino, thio, carbonyl and carboxyl groups, providing in the liquid reaction medium an enzyme and a substrate for the enzyme and

allowing reaction of the substrate to occur.

By tailoring the ion to comprise these functional groups, it is possible to carry out, in ionic liquid reaction media, enzyme-catalysed reactions that previously could not be carried out in non-aqueous environments.

The use of the defined ionic liquids for enzyme-catalysed reactions has several advantages over traditional organic solvents, biphasic systems and aqueous solutions. Ionic liquids have an ability to dissolve a wide range of inorganic, organic, polymeric and biological materials, often to a very high concentration. They have a wide liquid range, allowing both high and low temperature processes to be carried out in the same solvent. They do not elicit solvolysis phenomena and most stabilise short-lived reactive intermediates. There are no pH effects in the solvents and there is practically zero vapour pressure over much of the liquid range. Ionic liquids also exhibit excellent electrical and thermal conductivity whilst being non-flammable, recyclable and generally of low toxicity.

The use of the defined ionic liquids means that, for the first time it has been found possible to carry out a cofactor-dependent enzyme-catalysed reaction in an environment practically devoid of water.

Thus according to a second aspect of the invention, there is provided a method of carrying out a cofactor-dependent enzyme-catalysed reaction comprising

providing a liquid reaction medium which comprises an ionic liquid and less than 5% water,

providing in the liquid reaction medium a cofactor-dependent enzyme and the cofactor,

providing in the liquid reaction medium a substrate for the enzyme and

allowing reaction of the substrate to occur.

Preferably the level of water is very low, e.g. below 0.1%. Using ionic liquids as the solvent for these cofactor-dependent enzyme-catalysed reactions overcomes the problems associated with selecting a single solvent that is able to solvate the enzyme, the cofactor and the substrate, and that is also able to maintain activity of the enzyme.

According to a third aspect of the invention, there is provided a composition comprising

an ionic liquid including an ion which comprises a functional group selected from the group consisting of alkenyl, hydroxyl, amino, thio, carbonyl and carboxyl groups and an enzyme.

Such compositions allow enzyme-catalysed reactions to be carried out in a single phase system in which both the enzyme and the substrate are soluble, and in which the enzyme is active.

By “alkenyl” is meant any alkenyl group, preferably an alkenyl group with a carbon chain length of between 2 and 20 carbon atoms. The alkenyl group may be a straight chain, branched or a cyclic group.

By “ionic liquid” herein is meant a compound composed entirely of ions and which is liquid at the temperature at which the relevant reaction occurs, and preferably the ionic liquids have a melting point below 30° C., more preferably below 25° C. and most preferably below 20° C. They may even have melting point below 15° C. or below 10° C. If the enzyme being used can be active at relatively high temperatures, e.g. above 30° C., then the ionic liquid is simply required to be liquid at the reaction'temperature, but it is preferably liquid at 25° C. or below. Preferably the melting point of the ionic liquid is at least 10° C. below the temperature of the reaction.

The boiling point of the ionic liquid is preferably at least 200° C. It may be above 500° C. or even above 1100° C.

The ionic liquid includes an ion in which the functional group is selected from the group consisting of alkenyl, hydroxyl, amino, thio, carbonyl and carboxyl groups. Preferably the functional group is selected from the group consisting of hydroxyl, carbonyl and carboxyl. Most preferred is a functional group that is a hydroxyl group.

The ionic liquid may comprise more than one functional group selected from the group consisting of alkenyl, hydroxyl, amino, thio, carbonyl and carboxyl groups. If multiple functional groups are used they may be the same or different. We find that in some cases this can provide greater reactivity in the liquid reaction medium.

The choice of type of functional group and number of groups is dependent on the particular reaction under consideration. Using the method of the first aspect of the invention, it is possible to tailor the ionic liquid to a particular reaction, and thereby optimise the reaction conditions. The ionic liquid is selected so that both the enzyme and the substrate are soluble in the liquid reaction medium. It can be chosen also so that properties relevant to the reaction in question are optimised, for instance chirality, proticity, hydrogen-bonding, Lewis acidity and basicity, hydrophilicity, viscosity, gas solubilisation enthalpy, SHC etc.

It may be particularly useful to tailor the ionic liquid to mimic one or more properties of water. For example, ionic liquids may be tailored to have a labile proton, to be capable of hydrogen-bonding or to be polar.

Hydrogen-bonding moieties such as hydroxyl, amino and carboxyl groups may be particularly useful in reactions requiring a cofactor as they can assist in the solubilisation of the cofactor into the liquid reaction medium.

A polar moiety, for example a carbonyl, can also help with the dissolution of reaction components.

Moieties with a labile proton, such as hydroxyl, amino, thio and carboxyl groups can be especially useful. For example, in reactions in which the transfer of a proton is required, labile proton moieties can themselves provide a proton for the reaction.

By “labile proton” is meant herein a proton which has a pKₐ in the liquid reaction medium which is low enough to allow the proton to dissociate in the liquid reaction medium and take part in the enzyme-catalysed reaction, by allowing movement of protons between the ionic liquid, the substrate, the enzyme and the cofactor if present. Preferably the labile proton has a pKₐ of less than 25, more preferably between 10 and 20. Most preferably the pKₐ is about 15.

The method of the first aspect of the invention is particularly suited to reactions in which the enzyme requires a cofactor for activity. Cofactor-dependent enzymes have previously only been shown to be active in aqueous solvents due to the requirement for a polar, protic environment. Such an environment is necessary in order that the redox processes catalysed by these enzymes may occur.

The invention is particularly suited to those reactions in which the cofactors required in the cofactor-dependent enzyme-catalysed reactions are selected from nicotinamide nucleotides, flavin nucleotides and quinone cofactors. Particularly preferred are the nicotinamide cofactors NAD⁺ and NADP⁺ and their reduced counterparts NADH and NADPH.

Thus not only can the invention be used to provide a liquid reaction medium for commercially valuable cofactor-dependent enzyme-catalysed reactions, it can also be used to provide a medium for recycling cofactors which have been exhausted in a cofactor-dependent reaction carried out in another reaction medium which has not allowed recycling of the cofactor.

The method of the invention allows enzyme-catalysed reactions to be carried out in a liquid reaction medium comprising very low amounts of water. The liquid reaction medium preferably comprises less than 10% water, more preferably less than 5% water, more preferably less than 2% water and yet more preferably less than 1% water. It is especially preferred that the liquid reaction medium comprises less than 0.50% water, preferably less than 0.25% water and most preferably less than 0.10% water. In practice the invention is effective when the liquid reaction medium is substantially anhydrous and has e.g. less than 1000 ppm, preferably less than 100 ppm and more preferably less than 10 ppm water content. Water content at very low levels can be measured by NMR and Karl-Fischer titration. Water can elicit the hydrolysis of sensitive chemical species and it is therefore desirable to be able to avoid solvents comprising high levels of water if necessary.

Indeed, we find that in some cases enzyme activity is greater at levels of water below 0.1% than at levels above this. We have observed this. We have observed this, for instance, for hydroxy-substituted ionic liquids such as 1-(3-hydroxy-n-propyl)-3-methyl imidazolium hexafluorophosphate reaction media for enzyme reactions known to be effective in an aqueous medium, such as dehydrogenase reactions.

The liquid reaction medium used in the method of the invention can comprise very high levels of an ionic liquid. The liquid reaction medium may comprise other components but preferably comprises more than 90% ionic liquid, more, preferably more than 95% ionic liquid and yet more preferably more than 98% ionic liquid. It is especially preferred that the liquid reaction medium comprises more than 99.50% ionic liquid, more preferably more than 99.75% ionic liquid and yet more preferably more than 99.90% ionic liquid. It is most preferred that the liquid reaction medium consists of substantially 100% ionic liquid.

Thus in the invention the liquid reaction medium is generally a single-phase reaction medium.

The liquid reaction medium is a solvent for the reaction reagents. The liquid reaction medium may comprise very low levels of water, but this does not preclude the use of water as a reagent in a reaction carried out according to the method of the invention.

The ionic liquid used in all aspects of the invention may be made up of anions and cations or alternatively consist of zwitterions carrying both a positive and a negative charge on the same molecule. Most commonly the ionic liquid will comprise an anion and a cation. The ion comprising a functional group selected from the group consisting of an alkenyl, hydroxyl, amino, thio, carbonyl and carboxyl groups may be an anion, a cation or a zwitterion. Preferably it is a cation. If more than one functional group selected from the group consisting of alkenyl, hydroxyl, amino, thio, carbonyl and carboxyl groups is present then more than one group may be present on a single ion, e.g. the cation, but it is possible, alternatively, to include one or more functional groups on one ion and one or more functional groups on a different ion.

The cations utilised in the ionic liquids of the invention are typically compopsed of a quaternary nitrogen-based ion, preferably based on a nucleus selected from quaternary ammonium cations, pyrazolium cations, imidazolium cations, triazolium cations, pyridinium cations, pyridazinium cations, pyrimidinium cations, pyrazinium cations and triazinium cations. The heterocyclic nucleus may be substituted at any carbon or nitrogen atom by any alkyl, alkenyl, alkoxy, alkenedioxy, allyl, aryl, arylalkyl, aryloxy, amino, aminoalkyl, thio, thioalkyl, hydroxyl, hydroxyalkyl, oxoalkyl, carboxyl, carboxyalkyl, haloalkyl or halide function including all salts, ethers, esters, pentavalent nitrogen or phosphorus derivatives or stereoisomers thereof. When required and where possible, any of these functions may include a functional group selected from the group consisting of alkenyl, hydroxyl, amino, thio, carbonyl and carboxyl groups.

Preferred cations are those based on an imidazolium heterocyclic nucleus. Particularly preferred are those cations based on 1,3-disubstituted imidazolium.

The anions utilised in the ionic liquids of the invention may be of any type. The only theoretical constraint upon the choice of the anion is its ionic weight in order to keep the melting point of the ionic liquid below the desired temperature.

Preferably the anion is selected from halogenated inorganic anions, nitrates, sulfates, carbonates, sulfonates and carboxylates. The alkyl groups of the sulfonates and carboxylates may be selected from C₁-C₂₀ alkyl groups and may be substituted at any position with any alkyl, alkenyl, alkoxy, alkeneoxy, aryl, arylalkyl, aryloxy, amino, aminoalkyl, thio, thioalkyl, hydroxyl, hydroxyalkyl, carbonyl, oxoalkyl, carboxyl, carboxyalkyl or halide function, including all salts, ethers, esters, pentavalent nitrogen or phosphorus derivatives or stereoisomers thereof. For example, the anion may be selected from chloride, hexafluorophosphate, tetrafluoroborate, trifluoroacetate, methanesulfonate, glycolate, benzoate, salicylate, (±)-lactate, (+)-lactate, (−) lactate, (+)-pantothenate, (±)-tartrate, (+)-tartrate, (−)-tartrate, (±)-hydrogen tartrate, (+)-hydrogen tart rate, (−)-hydrogen tartrate, (±)-potassium tartrate, potassium tartrate, (−)-potassium tartrate, meso-tartrate, meso-1-hydrogen tartrate, meso-2-hydrogen tartrate, meso-1-potassium tartrate, meso-2-potassium tartrate. Chloride and other halide anions are less preferred, especially where the functional group on the cations is a hydroxyl, such as in 1-(3-hydroxy-n-propyl)-3-methyl imidazolium. An especially preferred anion is an organic carboxylate. When the anion is required to include a labile proton then tartrate and lactate functional groups are preferred. Tartrate, glycolate and lactate incorporate acid and hydroxyl functional groups. When the anion is not required to include one of the defined functional groups, hexafluorophosphate is preferred.

The enzyme used in the invention may be any type of enzyme. For instance it can be selected from oxidoreductases, hydrolases, dehydrogenases and lyases. The first aspect of the invention is particularly suitable for reactions in which the enzyme would be essentially inactive in an ionic liquid of the conventional type, namely those not comprising a functional group selected from the group consisting of alkenyl, hydroxyl, amino, thio, carbonyl and carboxyl groups. In the second aspect the enzyme is cofactor-dependent.

The methods of the invention are carried out under any conditions suitable for the reactants, enzyme and catalyst if used. Temperature can for instance be from about 10° C. to about 40° C. but is preferably from about 15° C. to about 25° C. Generally they are carried out at atmospheric pressure and ambient temperature.

The ionic liquids of the invention can be synthesised using methods-adapted from the general methods of Koel (see M. Koel, “Physical and Chemical Properties of Ionic Liquids Based on the Dialkylimidazolium Cation”, Proc. Estonian Akad. Sci. Chem., 2000, 49 (3)., 145-155), and Fuller (see J. Fuller, R. T. Carlin, H. C. de Long and D. Haworth, “Structure of 1-Ethyl-3-Methylimidazolium Hexafluorophosphate: Model for Room Temperature Molten Salts”, J. Chem. Soc., Them. Comm., 1994, 299-300). Equimolar amounts of a heterocyclic amine and the relevant alkyl halide are refluxed together for an extended period to generate the corresponding halide of the requisite cation. A metal carbonate is reacted with the acid precursor of the desired anion in order to generate the corresponding metal salt, which, is then dissolved or suspended in water whilst the aforementioned, halide is added in aqueous solution. After several hours stirring, the metal halide (if insoluble) is removed by filtration and the ionic liquid is purified (by solvent extraction to remove soluble metal halide if necessary) and dried prior to analysis by ¹H-NMR and UV-VIS/FT-IR spectrophotometry.

The ionic liquids may also be synthesised by first refluxing the relevant chloro-alcohol (or other appropriate halo-alcohol) with the chosen heterocyclic amine to generate a derivatised chloride (or other halide) salt. This can then be dissolved in water before adding silver (I) oxide (or other metal oxide leading to an insoluble halide) and removing the insoluble salts to give the hydroxide. The ammonium salt of choice can then be generated by reaction with the relevant acid.

Methods of synthesising ionic liquids are also disclosed in “Preparation and Characterization of New Room Temperature Ionic-Liquids”, Luis C. Branco et al., Chem. Eur. J., 2002, 8, 3671-3677 and “Ion conduction in zwitterionic-type molten salts and their polymers”, Yoshizawa et al., J. Mater. Chem., 2001, 11, 1057-1062, and other suitable methods may be used.

The invention will now be illustrated with reference to the following, examples in which BMIm⁺ is 1-butyl-3-methylimidazolium, HOPMIm⁺ is 1-(3-hydroxypropyl)-3-methylimidazolium and DHOPMIm⁺ is 1-(2,3-dihydroxypropyl)-3-methylimidazolium.

### EXAMPLE 1

### NAD⁺-dependent Alcohol Dehydrogenase from Bakers' Yeast (ADH1)

The oxidation of methanol to formaldehyde by ADH1, with concomitant reduction of NAD⁺ to NADH was examined. ADH1 (2 mg), NAD⁺ (50 mg) and methanol (10 μL) were dissolved in 10 mL volumes of the following solvents:

BMIm⁺PF₆⁻ (anhydrous by ¹H-NMR)

BMIm⁺PF₆⁻ (containing c. 0.5% v/v H₂O)

3-HOPMIm⁺PF₆⁻ (anhydrous by ¹H-NMR)

Aqueous pH7 dibasic potassium phosphate buffer.

Controls were set up in each solvent in which EITHER the cofactor OR the enzyme was omitted. The reaction vessels were stirred @ 25° C. for 24 hours under an argon atmosphere 50 μL samples were taken @ time points of 0,1,2,4,8,12 & 24 hours. The samples were assayed by adding 50 μL of chromotropic acid test reagent, diluting to 1 mL volume and monitoring the absorbance intensity of the formaldehyde-chromotropic acid adduct on an ultraviolet/visible spectrophotometer.

Results

Results are given in terms of the amount of formaldehyde produced relative to the aqueous standard. 0 indicates no activity, (−) indicates a lower amount, (±) an approximately equal result and (+) an increased amount.

| Solvent | 0 hrs | 1 hr | 2 hrs | 4 hrs | 8 hrs | 12 hrs | 24 hrs |
|---|---|---|---|---|---|---|---|
| BMIm⁺PF₆⁻ | 0 | 0 | 0 | 0 | (−) | (−) | (−) |
| (dry) | | | | | | | |
| BMIm⁺PF₆⁻ | 0 | (−) | (−) | (±) | (±) | (±) | (±) |
| (wet) | | | | | | | |
| 3-HOPMIm⁺PF₆⁻ | 0 | (±) | (±) | (±) | (±) | (±) | (+) |
| (dry) | | | | | | | |

Trace amounts of formaldehyde were detected in the controls, probably due to the slow oxidation of methanol to formaldehyde by the sulphuric acid component of the colour assay. However in no case did this approach the levels of formaldehyde detected in the experimental reaction samples.

### EXAMPLE 2

### NADP⁺-dependent Alcohol Dehydrogenase from Thermoanaerobium brockii (ADH2)

A second alcohol dehydrogenase, which utilises NADP⁺ as a cofactor, was examined. ADH2 (2 mg), NADP⁺ (250 mg) and 2-propanol (100 μL) were dissolved in 10 mL volumes of the following solvents:

BMIm⁺PF₆⁻ (anhydrous by ¹H-NMR)

BMIm⁺PF₆⁻ (containing c. 0.5% v/v H₂O)

3-HOPMIm⁺PF₆⁻ (anhydrous by ¹H-NMR)

Controls were set up in each solvent in which EITHER the cofactor OR the enzyme was omitted. The reaction vessels were stirred @ 25° C. for 24 hours under an argon atmosphere. 1 mL samples were taken @ time points of 0,2,4,12 & 24 hours. The samples were analysed by comparing the relative intensities of the characteristic ¹H-NMR shifts of the methyl group protons in 2-propanol and acetone. The results were confirmed in the case of the ionic liquid experiments by measuring the infra-red absorption intensities of the substrate and product by solution-phase FT-IR spectrophotometry.

Results

Results are given in terms of the amount of acetone produced with respect to the amount of 2-propanol remaining. 0 indicates that no acetone was detected, (−) denotes a smaller amount of acetone than of 2-propanol, (±) denotes an approximately equal concentration of acetone and 2-propanol and (+) denotes a greater amount of acetone than of 2-propanol.

| Solvent | 0 hrs | 2 hrs | 4 hrs | 12 hrs | 24 hrs |
|---|---|---|---|---|---|
| BMIm⁺PF₆⁻ (dry) | 0 | 0 | 0 | 0 | (−) |
| BMIm⁺PF₆⁻ (wet) | 0 | (−) | (−) | (−) | (±) |
| 3-HOPMIm⁺PF₆⁻ | 0 | (−) | (−) | (±) | (±) |
| (dry) | | | | | |

In the controls, no acetone was detected in any case.

The reverse reaction (reduction of acetone to 2-propanol) was also investigated. ADH2 (2 mg), NADPH (tetrasodium salt, 300 mg) and acetone (20 μL) were dissolved in 10 mL volumes of the following solvents:

BMIm⁺PF₆⁻ (anhydrous by ¹H-NMR)

BMIm⁺PF₆⁻(containing c. 0.5% v/v H₂O)

3-HOPMIm⁺PF₆⁻ (anhydrous by ¹H-NMR)

Controls were set up in each solvent in which EITHER the cofactor OR the enzyme was omitted. The reaction vessels were stirred @ 25° C. for 24 hours under an argon atmosphere. 1 mL samples were taken @ time points of 0,2,4,12 & 24 hours. The samples were treated and analysed by the techniques described above.

Results

Results are given in terms of the amount of 2-propanol produced with respect to the amount of acetone remaining.

0 indicates that no 2-propanol was detected, (−) denotes a smaller amount of 2-propanol than of acetone, (±) denotes an approximately equal concentration of 2-propanol and acetone and (+) denotes a greater amount of, product than of substrate.

| Solvent | 0 hrs | 2 hrs | 4 hrs | 12 hrs | 24 hrs |
|---|---|---|---|---|---|
| BMIm⁺PF₆⁻ (dry) | 0 | 0 | 0 | 0 | (−) |
| BMIm⁺PF₆⁻ (wet) | 0 | (−) | (−) | (−) | (−) |
| 3-HOPMIm⁺ PF₆⁻ | 0 | (−) | (−) | (−) | (±) |
| (dry) | | | | | |

In the controls, no 2-propanol was detected in any case.

### EXAMPLE 3

### NADP⁺-dependent Morphine Dehydrogenase from Pseudomonas putida M10 (MDH)

In protic solution, codeinone exists in dynamic equilibrium with its isomer neopinone; hence the two compounds are considered together.

Results—Oxidation of Codeine.

MDH (2 mg), NADP⁺ (50 mg) and codeine (free base, 20 mg) were dissolved in 10 mL volumes of the following solvents:

BMIm⁺PF₆⁻ (anhydrous by ¹H-NMR)

BMIm⁺PF₆⁻ (containing c. 0.5% v/v H₂O)

3-HOPMIm⁺PF₆⁻ (anhydrous by ¹H-NMR)

Aqueous pH7 dibasic potassium phosphate buffer.

Controls were set up in each solvent in which EITHER the cofactor OR the enzyme was omitted. The reaction vessels were stirred @ 25° C. for 24 hours under an argon atmosphere. 1 mL samples were taken @ time points of 0,1,2,4,8,12 & 24 hours. The samples were assayed by extraction with 0.5 mL CDCl₃ and analysis by ¹H-NMR spectroscopy, followed by removal of the solvent in vacuo and analysis of the solid residue by FT-IR spectrophotometry (KBr disc method). Results are given in terms of the amount of codeinone/neopinone produced relative to the amount of codeine remaining. 0 indicates that no codeinone/neopinone was detected, (−) denotes a smaller amount of codeinone-neopinone than of codeine, (±) denotes an approximately equal quantity of codeinone-neopinone and codeine and (+) denotes a greater amount of codeinone/neopinone than of codeine.

| Solvent | 0 hrs | 1 hr | 2 hrs | 4 hrs | 8 hrs | 12 hrs | 24 hrs |
|---|---|---|---|---|---|---|---|
| BMIm⁺PF₆⁻ | 0 | 0 | 0 | 0 | 0 | 0 | (−) |
| (dry) | | | | | | | |
| BMIm⁺PF₆⁻ | 0 | (−) | (−) | (−) | (−) | (−) | (±) |
| (wet) | | | | | | | |
| 3-HOPMIm⁺PF₆⁻ | 0 | (−) | (−) | (−) | (−) | (−) | (±) |
| (dry) | | | | | | | |
| Phosphate | 0 | (−) | (−) | (±) | (±) | (±) | (±) |
| buffer | | | | | | | |

In the controls, no codeinone/neopinone was detected in any case.

Results—Reduction of Codeinone.

MDH (2 mg), NADPH (tetrasodium salt, 55 mg) and codeinone (free base, 20 mg) were dissolved in 10 mL volumes of the following solvents:

BMIm⁺PF₆⁻ (anhydrous by ¹H-NMR)

BMIm⁺PF₆⁻ (containing c. 0.5% v/v H₂O)

3-HOPMIm⁺PF₆⁻ (anhydrous by ¹H-NMR)

Aqueous pH7 dibasic potassium phosphate buffer

Controls were set up in each solvent in which EITHER the cofactor OR the enzyme was omitted. The reaction vessels were stirred @ 25° C. for 24 hours under an argon atmosphere. 1 mL samples were taken @ time points of 0,1,2,4,8,12 & 24 hours. The samples were assayed by extraction with 0.5 mL CDCl₃ and analysis by ¹H-NMR spectroscopy, followed by removal of the solvent in vacuo and analysis of the solid residue by FT-IR spectrophotometry (KBr disc method). Results are given in terms of the amount of codeine produced relative to the amount of codeinone/neopinone remaining. 0 indicates that no codeine was detected, (−) denotes a smaller amount of codeine than of codeinone/neopinone, (±) denotes a approximately equal quantity of codeine and codeinone/neopinone and (+) denotes a greater amount of codeine than of codeinone/neopinone.

| Solvent | 0 hrs | 1 hr | 2 hrs | 4 hrs | 8 hrs | 12 hrs | 24 hrs |
|---|---|---|---|---|---|---|---|
| BMIm⁺PF₆⁻ | 0 | 0 | 0 | 0 | 0 | (−) | (−) |
| (dry) | | | | | | | |
| BMIm⁺PF₆⁻ | 0 | (−) | (−) | (±) | (+) | (+) | (+) |
| (wet) | | | | | | | |
| 3-HOPMIm⁺PF₆⁻ | 0 | (−) | (±) | (+) | (+) | (+) | (+) |
| (dry) | | | | | | | |
| Phosphate | 0 | (−) | (+) | (+) | (±) | (±) | (±) |
| buffer | | | | | | | |

In the controls, some spontaneous reduction of codeinone/neopinone was observed. However this was in no case of comparable extent to that achieved in the experimental reaction samples. The isomerisation between codeinone and neopinone was not observed in dry BMIm⁺PF₆⁻ but was seen in all other samples, being most pronounced in the aqueous systems. The spontaneous breakdown of codeinone became a factor in the later samples but appeared to be hindered by the ionic liquids. Spontaneous breakdown of codeinone in dry BMIm⁺PF₆⁻ was negligible even after 24 hours.

### EXAMPLE 4

### Recycling NADP⁺ using MDH and ADH2

Results—Oxidation of Codeine.

In the first instance, the oxidation of codeine to codeinone by MDH, with reduction of NADP⁺ to NADPH, was coupled with the ADH2-mediated reduction of acetone to 2-propanol, in order to recycle the NADP⁺. MDH (2 mg), ADH2 (2 mg), NADP⁺ (25 mg), codeine (free base, 440 mg) and acetone (500 μL) were dissolved in 10 mL of the following solvents:

BMIm⁺PF₆⁻ (anhydrous by ¹H-NMR)

BMIm⁺PF₆⁻ (containing c. 0.5% v/v H₂O)

3-HOPMIm⁺PF₆⁻ (anhydrous by ¹H-NMR)

Aqueous pH7 dibasic potassium phosphate buffer.

Controls were set up in each solvent in which EITHER the cofactor OR the enzyme was omitted. The reaction vessels were stirred @ 25° C. for 24 hours under an argon atmosphere. 1 mL samples were taken @ time points of 0,1,2,4,8,12 & 24 hours. The samples were assayed by extraction with 0.5 mL CDCl₃ and analysis by ¹H-NMR spectroscopy, followed by removal of the solvent in vacuo and analysis of the solid residue by FT-IR spectrophotometry (KBr disc method). Results are given in terms of the amount of codeine produced relative to the amount of codeine remaining. 0 indicates that no codeinone/neopinone was detected, (−) denotes a smaller amount of codeine than of codeinone/neopinone than of codeine, (±) denotes a approximately equal quantity of codeinone/neopinone and codeine and (+) denotes a greater amount of codeinone/neopinone than of codeine.

| Solvent | 0 hrs | 1 hr | 2 hrs | 4 hrs | 8 hrs | 12 hrs | 24 hrs |
|---|---|---|---|---|---|---|---|
| BMIm⁺PF₆⁻ | 0 | 0 | 0 | 0 | 0 | 0 | (−) |
| (dry) | | | | | | | |
| BMIm⁺PF₆⁻ | 0 | (−) | (−) | (−) | (−) | (±) | (±) |
| (wet) | | | | | | | |
| 3-HOPMIm⁺PF₆⁻ | 0 | (−) | (±) | (±) | (±) | (±) | (+) |
| (dry) | | | | | | | |
| Phosphate | 0 | (−) | (±) | (±) | (±) | (+) | (+) |
| buffer | | | | | | | |

In the controls, no codeinone/neopinone was detected in any case. In view of the large excess of acetone incorporated into the reaction mixtures (in order to force the equilibrium of the MDH-mediated reaction in favour of codeinone/neopinone), no attempt was made to quantify the amount of 2-propanol produced. The appearance of codeinone/neopinone in all reaction mixtures (except dry BMIm⁺PF₆⁻) was accompanied by the concomitant appearance of the orange coloration seen with high concentrations of codeinone/neopinone in ionic liquids. On prolonged standing at room temperature after the last sample had been taken, codeinone/neopinone began to precipitate from the remaining 3-HOPMIm⁺PF₆⁻ reaction solution.

Results—Reduction of Codeinone.

MDH (2 mg), ADH2 (2 mg), NADPH (tetrasodium salt, 30 mg), codeinone (free base, 425 mg) and 2-propanol (500 μL) were dissolved in 10 mL of the following solvents:

BMIm⁺PF₆⁻ (anhydrous by ¹H-NMR)

BMIm⁺PF₆⁻ (containing c. 0.5% v/v H₂O)

3-HOPMIm⁺PF₆⁻ (anhydrous by ¹H-NMR)

Aqueous pH7 dibasic potassium phosphate buffer.

Controls were set up in each solvent in which EITHER the cofactor OR the enzyme was omitted. The reaction vessels were stirred @ 25° C. for 24 hours under an argon atmosphere. 1 mL samples were taken @ time points of 0,1,2,4,8,12 & 24 hours. The samples were assayed by extraction with 0.5 mL CDCl₃ and analysis by ¹H-NM spectroscopy, followed by removal of the solvent in vacuo and analysis of the solid residue by FT-IR spectrophotometry (KBr disc method). Results are given in terms of the amount of codeine produced relative to the amount of codeinone/neopinone remaining. 0 indicates that no codeine was detected, (−) denotes a smaller amount of codeine than of codeinone/neopinone, (±) denotes an approximately equal quantity of codeine and codeinone/neopinone and (+) denotes a greater amount of codeine than of codeinone/neopinone.

| Solvent | 0 hrs | 1 hr | 2 hrs | 4 hrs | 8 hrs | 12 hrs | 24 hrs |
|---|---|---|---|---|---|---|---|
| BMIm⁺PF₆⁻ | 0 | 0 | 0 | 0 | 0 | (−) | (−) |
| (dry) | | | | | | | |
| BMIm⁺PF₆⁻ | 0 | (−) | (−) | (−) | (±) | (±) | (±) |
| (wet) | | | | | | | |
| 3-HOPMIm⁺PF₆⁻ | 0 | (−) | (±) | (±) | (±) | (+) | (+) |
| (dry) | | | | | | | |
| Phosphate | 0 | (−) | (±) | (±) | (+) | (+) | (+) |
| buffer | | | | | | | |

In the controls, some spontaneous reduction of: codeinone/neopinone was observed. However this was in no case of comparable extent to that achieved in the experimental reaction samples. The isomerisation between codeinone and neopinone was again not observed in dry BMIm⁺PF₆⁻ but was seen in all other samples, being most pronounced in the aqueous systems. The spontaneous breakdown of codeinone again became a factor in the later samples but appeared to be hindered by the ionic liquids. Spontaneous breakdown of codeinone in dry BMIm⁺PF₆⁻ was negligible even after 24 hours.

In all of the above experiments, equal concentrations of the substrate were used in both ionic and aqueous solutions, for the purposes of direct comparison. However, it should be noted that ionic liquids facilitate the dissolution of organic substrates to concentrations far in excess of those used above (and of those attainable in aquo) and that consequently the rates and turnovers which may theoretically be achieved by using biocatalysis in ionic liquids are significantly higher than those currently attainable in aquo.

The synthesis of the ionic liquid materials used in these examples may be carried out according to the method outlined below in Example 7.

### EXAMPLE 5

### NADP⁺-dependent Morphine Dehydrogenase from Pseudomonas putida M10 (MDH)

Results—Oxidation of Codeine.

MDH (2 mg), NADP⁺ (50 mg) and codeine (free base, 20 mg) were dissolved in 10 mL of racemic 1-(2,3-dihydroxypropyl)-3-methylimidazolium hexafluorophosphate ((±)-DHOPMIm PF⁶), which was anhydrous by both ¹H-NMR and Karl Fischer titration. An identical reaction was set-up in pH7 aqueous phosphate buffer.

Controls were set up in each solvent in which EITHER the cofactor OR the enzyme was omitted. The reaction vessels were stirred @ 25° C. for 24 hours under an argon atmosphere. 1 mL samples were taken @ time points of 0,1,2,4,8,12 & 24 hours. The samples were assayed by extraction with 0.5 mL CDCl₃ and analysis by ¹H-NMR spectroscopy, followed by removal of the solvent in vacuo and analysis of the solid residue by FT-IR spectrophotometry (KBr disc method). Results are given in terms of the amount of codeinone/neopinone produced relative to the amount of codeine remaining. 0 indicates that no codeinone/neopinone was detected, (−) denotes a smaller-amount of codeinone/neopinone than of codeine, (±) denotes an approximately equal quantity of codeinone/neopinone and codeine and (+) denotes a greater amount of products than of substrate.

| Solvent | 0 hrs | 1 hr | 2 hrs | 4 hrs | 8 hrs | 12 hrs | 24 hrs |
|---|---|---|---|---|---|---|---|
| rac- | 0 | (−) | (−) | (−) | (−) | (±) | (±) |
| DHOPMIm⁺ | | | | | | | |
| PF₆⁻ (dry) | | | | | | | |
| Phosphate | 0 | (−) | (−) | (±) | (±) | (±) | (±) |
| buffer | | | | | | | |

In the controls, no codeinone/neopinone was detected in any case.

Results—Reduction of Codeinone.

The activity of the enzyme in the reverse direction was investigated to ensure applicability to the NADPH-dependent system. MDH (2 mg), NADPH (tetrasodium salt, 55 mg) and codeinone (free base, 20 mg) were dissolved in 10 mL volumes of the above solvents.

Controls were set up in each solvent in which EITHER the cofactor OR the enzyme was omitted. The reaction vessels were stirred @ 25° C. for 24 hours under an argon atmosphere. 1 mL samples were taken @ time points of 0,1,2,4,8,12 & 24 hours. The samples were assayed by extraction with 0.5 mL CDCl₃ and analysis by ¹H-NMR spectroscopy, followed by removal of the solvent in vacuo and analysis of the solid residue by FT-IR spectrophotometry (KBr disc method). Results are given in terms of the amount of codeine produced relative to the amount of codeinone/neopinone remaining. 0 indicates that no codeine was detected, (−) denotes a smaller amount of codeine than of codeinone/neopinone, (±) denotes an approximately equal quantity of codeine and codeinone/neopinone and (+) denotes a greater amount of product than of substrate,

| Solvent | 0 hrs | 1 hr | 2 hrs | 4 hrs | 8 hrs | 12 hrs | 24 hrs |
|---|---|---|---|---|---|---|---|
| rac- | 0 | (−) | (−) | (±) | (±) | (±) | (±) |
| DHOPMIm⁺ | | | | | | | |
| PF₆⁻ (dry) | | | | | | | |
| Phosphate | 0 | (−) | (−) | (±) | (±) | (±) | (±) |
| buffer | | | | | | | |

In the controls, some spontaneous reduction of codeinone/neopinone was observed. This was in no case comparable to that achieved in the experimental reaction samples. The isomerisation between codeinone and neopinone was observed in all samples. The spontaneous breakdown of codeinone was greater in DHOPMIm than in the comparable HOPMIm reaction, but much less significant than in aquo.

### EXAMPLE 6

### Recycling NADP⁺ using MDH and ADH

Results—Oxidation of Codeine.

The oxidation of codeine to codeinone by MDH, with reduction of NADP⁺ to NADPH, was coupled with the ADH-mediated reduction of acetone to 2-propanol, in order to recycle the NADP⁺. MDH (2 mg), ADH2 (2 mg), NADP⁺ (25 mg), codeine (free base, 440 mg) and acetone (50 μL) were dissolved in 10 mL of the above solvents.

Controls were set up in each solvent in which EITHER the cofactor OR the enzyme was omitted. The reaction vessels were stirred @ 25° C. for 24 hours under an argon atmosphere. 1 mL samples were taken @ time points of 0,1,2,4,8,12 & 24 hours. The samples were assayed by extraction with 0.5 mL CDCl₃ and analysis by ¹H-NMR spectroscopy, followed by removal of the solvent in vacuo and analysis of the solid residue by FT-IR spectrophotometry (KBr disc method). Results are given in terms of the amount of codeinone/neopinone produced relative to the amount of codeine remaining. 0 indicates that no codeinone/neopinone was detected, (−) denotes a smaller amount of codeinone/neopinone than of codeine.

(±) denotes an approximately equal quantity of codeinone/neopinone and codeine and (+) denotes a greater amount of products than of substrate.

| Solvent | 0 hrs | 1 hr | 2 hrs | 4 hrs | 8 hrs | 12 hrs | 24 hrs |
|---|---|---|---|---|---|---|---|
| rac- | 0 | (−) | (±) | (±) | (±) | (+) | (+) |
| DHOPMIm⁺ | | | | | | | |
| PF₆⁻ (dry) | | | | | | | |
| Phosphate | 0 | (−) | (±) | (±) | (±) | (+) | (+) |
| buffer | | | | | | | |

In the controls, no codeinone/neopinone was detected in any case. In view of the large excess of acetone incorporated into the reaction mixtures (in order to force the equilibrium of the MDH-mediated reaction in favour of codeinone/neopinone), no attempt was made to quantify the amount of 2-propanol produced. The appearance of codeinone/neopinone in reaction mixtures was accompanied by the concomitant appearance of the orange coloration seen with high concentrations of codeinone/neopinone in ionic liquids.

Results—Reduction of Codeinone.

MDH (2 mg), ADH2 (2 mg), NADPH (tetrasodium salt, 30 mg), codeinone (free base, 425 mg) and 2-propanol (500 μL) were dissolved in 10 mL of the above solvents.

Controls were set up in each solvent in which EITHER the cofactor OR the enzyme was omitted. The reaction vessels were stirred @ 25° C. for 24 hours under an argon atmosphere. 1 mL samples were taken @ time points of 0,1,2,4,8,12 & 24 hours. The samples were assayed by extraction with 0.5 mL CDCl₃ and analysis by ¹H-NMR spectroscopy, followed by removal of the solvent in vacuo and analysis of the solid residue by FT-IR spectrophotometry (KBr disc method). Results are given in terms of the amount of codeine produced relative to the amount of codeinone/neopinone remaining. 0 indicates that no codeine was detected, (−) denotes a smaller amount of codeine than of codeinone/neopinone, (±) denotes an approximately equal quantity of codeine and codeinone/neopinone and (+) denotes a greater amount of product than of substrate.

| Solvent | 0 hrs | 1 hr | 2 hrs | 4 hrs | 8 hrs | 12 hrs | 24 hrs |
|---|---|---|---|---|---|---|---|
| rac- | 0 | (−) | (±) | (±) | (±) | (+) | (+) |
| DHOPMIm⁺ | | | | | | | |
| PF₆⁻ (dry) | | | | | | | |
| Phosphate | 0 | (−) | (±) | (±) | (+) | (+) | (+) |
| buffer | | | | | | | |

In the controls, some spontaneous reduction of codeinone/neopinone was observed. This was not comparable to that achieved in the experimental reaction samples. The isomerisation between codeinone and neopinone was observed in all samples. The spontaneous breakdown of codeinone was greater in DHOPMIm than in the comparable HOPMIm reaction, but much less significant than in aquo. In all of the above experiments, equal concentrations of, the substrate were used in both ionic and aqueous solutions, for the purposes of direct comparison. The concentration enhancement strategy previously noted for HOPMIm etc. is again applicable here.

The incorporation of a second labile protic group into the ionic liquid results in an improvement in enzyme activity with respect to HOPMIm. However, the differences in enzyme activity observed between HOPMIm and DHOPMIm are much less significant than those between HOPMIm and BMIm.

Additional note—when the above biotransformations were attempted in the anhydrous 2-methyl capped form of BMIm (with no labile acidic proton), no transformations over and above background levels were observed.

### EXAMPLE 7

### Synthesis of 1-(3-hydroxypropyl)-3-methylimidazolium hexafluorophosphate (3-HOPMIm PF₆)

### (a) Preparation of 1-(3-hydroxypropyl)-3-methylimidazolium chloride

3-Chloro-1-propanol (Lancaster; 0.26 moles, 24.58 g) and 1-methylimidazole (Lancaster, 1 equiv., 21.35 g) were refluxed for 36 hours in a round-bottomed flask equipped with magnetic stirrer and reflux condenser. The resultant viscous yellow liquid was cooled to room temperature and washed three times with dry ether. The washings were discarded and the product was heated to 50° C. in vacuo for 8 hours to remove residual solvent. Recovered material was a viscous pale yellow liquid, density 1.12 g cm⁻³ @ 25° C., yield 41.9 g=91%.

NMR (1H, 400 MHz, D₂O) 1.99 (q, 2H, β-CH₂), 3:54 (t, 2H, α-CH₂), 4.02 (s, 3H, N—CH₃), 4.65 (t, 2H, γ-CH₂), 7.90 (d, 1H, H-4), 8.10 (d, 1H, H-5), 9.55 (s, 1H, H-2).

### (b) Preparation of 1-(3-hydroxypropyl)-3-methylimidazolium hydroxide

1-(3-Hydroxypropyl-3-methylimidazolium chloride was dissolved in water to a final concentration of 2.20M (36.69 g in 93 mL). Silver (I) oxide (Aldrich, 0.5 equiv, 24.1 g) was added in the solid state and the reaction was stirred in the dark at room temperature for 24 hours. At the conclusion of this period, the insoluble silver salts were removed by filtration and the highly basic aqueous solution was concentrated by heating to 50° C. in vacuo for 5 hours. The resultant gummy material was extremely hygroscopic and was used immediately in the next step.

### (c) Preparation of 1-(3-hydroxypropyl)-3-methylimidazolium hexafluorophosphate

1-(3-Hydroxypropyl)-3-methylimidazolium hydroxide (30.2 g, from above) was dissolved in 50 mL water in a plastic beaker. Hexafluorophosphoric acid (Aldrich, 49 mL of 60 wt. % soln.) was added dropwise via the dropping funnel, with stirring, until the mixture was no longer basic. The reaction vessel was cooled in ice for the duration of the reaction and for 1 hour thereafter. The pale yellow solution was then stirred at room temperature for 12 hours. At the conclusion of this period the product solution was dried under vacuum at 50° C. for 8 hours. The crude product was dissolved in dry acetonitrile and treated with activated charcoal to remove coloured impurities. The product was then chromatographed on a basic alumina column using dry acetonitrile as the eluent, to remove chloride. To dry the product, the solvent was removed at 50° C. in vacuo and the resultant colourless liquid was frozen in liquid nitrogen, placed under high vacuum and gradually allowed to warm to room temperature. It was then heated to 80° C. and left under high vacuum at this temperature for 48 hours. Product was a colourless liquid, yield 39.4 g (68%).

NMR: (¹H, 400 MHz, d₆-DMSO, D₂O shake) 1.92 (q, β-CH₂), 3.40 (t, 2H, α-CH₂), 3.83 (s, 3H, N—CH₃), 4.21 (t, 2H, γ-CH₂), 7.65 (d, 1H, H-4), 7.72 (d, 1H, H-5), 9.13 (s, 1H, H-2).

FT-IR: Principal absorbances (Nujol mull) 3382, 1576, 1167, 871, 2963, 2892, 2094, 1634, 742, 624 cm⁻¹.

Chloride content not detectable by AgNO₃. Water content <10 ppm by Karl Fischer titration.

1. A method of carrying out an enzyme-catalysed reaction comprising
providing a liquid reaction medium which comprises an ionic liquid including an ion which comprises a functional group selected from the group consisting of alkenyl, hydroxyl, amino, thio, carbonyl and carboxyl groups,
providing in the liquid reaction medium an enzyme and a substrate for the enzyme,
and allowing reaction of the substrate to occur.

2. A method according to claim 1 in which the functional group is selected from the group consisting of hydroxyl, carbonyl and carboxyl groups.

3. A method according to claim 1 in which the functional group is a hydroxyl group.

4. A method according to claim 1 in which the functional group has a labile proton.

5. A method according to claim 4 in which the labile proton has a pKₐ of less than 25.

6. A method according to claim 1 in which the ionic liquid comprises more than one functional group selected from the group consisting of alkenyl, hydroxyl, amino, thio, carbonyl and carboxyl groups.

7. A method according to claim 1 in which the ionic liquid comprises either an anion and a cation or a zwitterion.

8. A method according to claim 1 in which the ion comprising a functional group is a cation.

9. A method according to claim 1 in which the enzyme requires a cofactor and said cofactor is provided in the liquid reaction medium.

10. A method according to claim 1 in which the liquid reaction medium comprises less than 1.00% water.

11. A method according to claim 1 in which the liquid reaction medium comprises at least 99.00% of the ionic liquid.

12. A method of carrying out a cofactor-dependent enzyme-catalysed reaction comprising
providing a liquid reaction medium which comprises an ionic liquid and less than 5% water,
providing in the liquid reaction medium a cofactor-dependent enzyme and the cofactor,
providing in the liquid reaction medium a substrate for the enzyme and
allowing reaction of the substrate to occur.

13. A method according to claim 12 in which the ionic liquid includes an ion which comprises a functional group selected from the group consisting of alkenyl, hydroxyl, amino, thio, carbonyl and carboxyl groups.

14. A method according to claim 13 in which the functional group is a hydroxyl group.

15. A method according to claim 12 in which the ionic liquid includes an ion which comprises a functional group which has a labile proton.

16. A method according to claim 15 in which the labile proton has a pKₐ of less than 25.

17. A method according to claim 12 in which the ionic liquid comprises more than one functional group selected from the group consisting of alkenyl, hydroxyl, amino, thio, carbonyl and carboxyl groups.

18. A method according to claim 12 in which the liquid reaction medium comprises less than 1.00% water, preferably.

19. A method according to claim 12 in which the liquid reaction medium comprises at least 99.00% of the ionic liquid.

20. A composition comprising
an ionic liquid including an ion which comprises a functional group selected from the group consisting of alkenyl, hydroxyl, amino, thio, carbonyl and carboxyl groups and
an enzyme.

21. A composition according to claim 20 in which the functional group is selected from the group consisting of hydroxyl, carbonyl and carboxyl groups.

22. A composition according to claim 21 in which the functional group is a hydroxyl group.

23. A composition according to claim 20 in which the functional group has a labile proton.

24. A composition according to claim 23 in which the labile proton has a pKₐ of less than 25.

25. A composition according to claim 20 which further comprises a substrate for the enzyme.

26. A composition according to claim 20 in which the enzyme requires a cofactor and the composition comprises said cofactor.

27. (canceled)

28. A method according to claim 1 in which the liquid reaction medium comprises less than 0.10% water.

29. A method according to claim 12 in which the liquid reaction medium comprises less than 0.1% water.

30. A method of carrying out an enzyme-catalysed reaction comprising the step of combining an enzyme with an ionic liquid that comprises an ion which comprises a functional group selected from the group consisting of alkenyl, hydroxyl, amino, thio, carbonyl and carboxyl groups.
